(19) United States
(12) Patent Application Publication
(10) Pub. No.: US 2025/0195886 A1
(43) Pub. Date: Jun. 19, 2025

(54) Wearable Device For Managing Vagus Nerve Stimulation

(71) Applicants: Laura M. Gustavson, Redmond, WA (US); Ronald K. Rowbotham, Lake Forest Park, WA (US); David P. Finch, Bothell, WA (US); Brian J. Bennet, Redmond, WA (US); Aron A. White, Kirkland, WA (US); Melinda J. Wang, Seattle, WA (US); Pamela F. Breske, Newcastle, WA (US)

(72) Inventors: Laura M. Gustavson, Redmond, WA (US); Ronald K. Rowbotham, Lake Forest Park, WA (US); David P. Finch, Bothell, WA (US); Brian J. Bennet, Redmond, WA (US); Aron A. White, Kirkland, WA (US); Melinda J. Wang, Seattle, WA (US); Pamela F. Breske, Newcastle, WA (US)

(21) Appl. No.: 18/983,173

(22) Filed: Dec. 16, 2024

Related U.S. Application Data
(60) Provisional application No. 63/677,951, filed on Jul. 31, 2024.
(60) Provisional application No. 63/610,998, filed on Dec. 15, 2023.

(51) Int. Cl. A61N 1/36; A61N 1/04; A61N 1/362; G16H 10/60; G16H 20/30; G16H 40/63

(52) U.S. Cl. CPC A61N 1/36034; A61N 1/0456; A61N 1/36025; A61N 1/36031; A61N 1/3625; G16H 10/60; G16H 20/30; G16H 40/63

(57) ABSTRACT

A medical management system is provided that includes components to monitor a patient and manage vagus nerve stimulation (VNS), such as transcutaneous VNS (tVNS). Patient data indicating patient parameters are captured by sensors of a wearable device intended for long-term wear by the patient. The resulting patient data are analyzed to gain information on the treatment and aspects of the person that may facilitate a closed loop treatment system to make adjustments, such as changes to timing factors for treatments, as well as making comprehensive decisions about treatment plans, such as foregoing one modality of treatment for another, discontinuing treatment, or augmenting with additional types of treatment.

### FIELD OF THE INVENTION

The present disclosure generally relates to use of a medical management system to manage vagus nerve stimulation (VNS) to provide therapy for health-related issues of a patient.

### CROSS REFERENCES TO RELATED APPLICATIONS

This application claims the benefit of the filing date of U.S. Provisional Patent Application No. 63/610,998, filed Dec. 15, 2023, and claims the benefit of the filing date of U.S. Provisional Patent Application No. 63/677,951, filed Jul. 31, 2024, the disclosures of which is hereby incorporated herein by reference for all purposes.

### BACKGROUND

Medical devices that are intended for a patient to wear, e.g., wearable devices, can be used to treat a patient and/or gather health related data on the patient. Particular wearable devices can provide electrical stimulation treatment by integrated electrical components or by controlling an external device. For example, electrodes can be used to emit electrical current into the body in a manner to benefit the patient.

Some treatment devices stimulate the vagus nerve (hereinafter, “VNS”) of a patient to care for various types of health issues, such as cardiac, neurological, autonomic disfunction, psychological, inflammatory, immune, pain-related, and other types of health concerns. Electrodes of such stimulation devices can be placed against the body, be worn by the patient, or be implanted in the patient in areas of the body in which the vagus nerve passes close to or is innervated by the vagus nerve. Some stimulation devices provide non-invasive transcutaneous electrical stimulation of the vagus nerve (hereinafter, “tVNS”). In some cases, a stimulation device is positioned at the ear of a patient for auricular stimulation, such as at the tragus part of the ear.

Stimulation of the vagus nerve is widely studied and used for the treatment of a variety of health issues, many of which have multiple pathophysiological mechanisms. As such, day to day occurrences of a patient can significantly impact treatment results and warrant changes to treatment plans.

A health issue that is treatable by vagus nerve stimulation is Postural Tachycardia Syndrome (POTS). See, for example, Stavrakis, Noninvasive Vagus Nerve Stimulation in Postural Tachycardia Syndrome, JACC: Clinical Electrophysiology, 2023 (hereinafter, “Stavrakis”), which is incorporated herein in its entirety for all purposes. Stavrakis states that, “POTS is a heterogeneous disease” (See, Stavrakis, pages 8, Study Limitations) and “treatment of POTS typically requires a personalized approach to each patient.” (See, Stavrakis, pages 6, Discussion, paragraph 1).

There is often a lack of universal predefined treatment guidelines for some ailments that may be addressable by tVNS, such as POTS and Long COVID, due to the complexity of such health issues. Responsiveness to treatments can vary and be impacted by numerous factors such as a patient state, for example, stress level, and patient actions, for example, diet, fluid intake, and exercise. Diligent oversight of tVNS treatments allows for greater control over outcomes and appropriate treatment regimens, measurements of success, and situation-base decision making for individual treatments and broader treatment plans.

### SUMMARY

A medical management system (also referred to as the “management system”) is provided that includes components to monitor a patient and manage VNS, such as tVNS and implanted VNS treatment. Data indicating patient parameters are captured by sensors of a wearable device of the management system that is intended for long-term wear by the patient. The resulting patient data are analyzed to gain information on the treatment and aspects of the person that may facilitate a closed loop treatment system to make adjustments, such as changes to timing factors for treatments, as well as making comprehensive decisions about treatment plans, such as foregoing one modality of treatment for another, discontinuing treatment, or augmenting with additional types of treatment.

The medical management system comprises a wearable article configured for long term wear on a patient and long term acquisition of patient data. The management system includes a control unit configured to direct a stimulation device to provide first VNS sessions according to a first treatment plan, one or more sensors configured to capture signals indicative of at least one patient parameter, and a data handling unit in communication with the one or more sensors to generate patient data from the signals related to the first treatment plan. An interface is also configured for transmitting the patient data, for example directly or via a patient communication device, for receipt by a remote data management device to conduct data analysis with respect to the first treatment plan. The interface is further configured for receiving a revision of the first treatment plan by the remote data management device. Such treatment plan revision may include a time factor change for treatment and/or a type of treatment change based, at least in part, on the data analysis. The control unit is further configured to direct the stimulation device according to the revision.

In some implementations, the patient data may include treatment data indicating delivery characteristics of the first VNS sessions. Data analysis may indicate a lack of patient compliance with the first treatment plan and revision to the treatment plan may include a change to the first treatment plan to address the lack of patient compliance.

At least a portion of the signals by the sensors may be captured during a post-treatment period after conclusion of the first treatment plan. The data analysis of the patient data from these signal portions may indicate a patient regression of health. The interface of the management system is further configured to receive a second treatment plan (for example, after the first treatment plan is discontinued or completed) from the data management device including instructions for: repeating the first VNS sessions, providing second VNS sessions using a different stimulation device, and/or providing a supplemental other treatment type.

The medical management system of claim 3, wherein the control unit is further configured to direct the repeating of the first VNS sessions by the stimulation device or the providing of second VNS sessions by the different stimulation device, according to the second treatment plan.

In still some implementations, a revision of the treatment plan specifies a particular change in a time factor that is determined based, at least in part, on the signals that indicate a patient state, patient action, and/or environment. For example, the time factor may include at least one of a VNS session duration, a time of day for at least one first VNS session, a period of time for a course of multiple VNS sessions, and discontinuing of the first VNS sessions.

Some implementations of the management system may include a communication component to transmit a notification of an impending session of the VNS first sessions prior to controlling the providing of the impending session.

The described management system components such as by a wearable medical device (WMD) may be for long term wear on a patient to manage vagus nerve stimulation (VNS) treatment for a health issue of a patient. The components, such as the WMD may be further configured to implement a method for receiving signals indicative of at least one patient parameter, captured by one or more sensors of the WMD; directing a stimulation device to provide first VNS sessions according to a first treatment plan; processing the signals to generate patient data; transmitting the patient data for receipt by a remote management device to conduct data analysis with respect to the first treatment plan; receiving a revision of the first treatment plan generated by the remote management device, the revision including a time factor change for treatment and/or type of treatment change based, at least in part, on the data analysis; and controlling the stimulation device according to the revision. The method may further include steps described above performed by various management system components.

Some medical management systems as described herein may be adapted and configured for managing transcutaneous vagus nerve stimulation (tVNS) for treatment of Postural Tachycardia Syndrome (POTS) of a patient. For example, a wearable article may be configured for long term wear on a torso of the patient including one or more sensors configured to capture signals indicative of at least one patient parameter including a patient state.

In such POTS treatment management systems, patient data may include a heartbeat rate and body position and a revision of the treatment plan of a treatment time factor may include a change in a session duration for treatment. The revision may also include a supplemental other treatment type including an exercise regime and the patient data includes body motion data by an accelerometer.

In some POTS treatment management systems, the WMD may further comprise cardiac pacing components and the treatment plan includes outputting pacing pulses to the patient by the cardiac pacing components when the patient is determined to be standing from a sitting or lying position.

In some implementations of the POTS treatment management system, at least a portion of the signals may be captured during a post-treatment period after conclusion of the first treatment plan. The data analysis may indicate a patient regression. In this case, the treatment plan revision may include instructions for: repeating the first tVNS sessions, providing second tVNS sessions using a different stimulation device, and/or providing a supplemental other treatment type.

A further understanding of the nature and the advantages of particular implementations disclosed herein may be realized by reference of the remaining portions of the specification and the attached drawings.

### BRIEF DESCRIPTION OF THE DRAWINGS

Various implementations in accordance with the present disclosure will be described with reference to the drawings.

FIG. 1 is an example medical management system having system components to manage tVNS treatment, in accordance with some implementations.

FIG. 2 is a block diagram of example functional components of the medical management system, in accordance with some implementations.

FIG. 3 is a schematic diagram of the example medical management system with a wearable article, control unit, and other components, in accordance with some implementations.

FIG. 4 is a schematic diagram of an example stimulation device in use on an ear, in accordance with some implementations.

FIGS. 5A and 5B are flowcharts of an example method for managing VNS treatment, in which FIG. 5A shows example steps performed by a WMD, and FIG. 5B shows example steps performed by a data management device, in accordance with some implementations.

FIG. 6 is a block diagram of an example WMD control unit and an example data management device, in accordance with some implementations.

### DETAILED DESCRIPTION

In the following description, various implementations will be described. For purposes of explanation, specific configurations and details are set forth in order to provide a thorough understanding of the implementations. However, it will also be apparent to one skilled in the art that the implementations may be practiced without the specific details. Well-known features may be omitted or simplified without obscuring the implementations described. The description of the medical management system provides a framework which can be tailored to individual systems built around the medical management system. Elements may be described in terms of “basic functionality” or varying degrees of functionality.

The present medical management system includes components to monitor a patient and to oversee and control VNS treatment of a health issue. The management system includes a wearable medical device (also referred to as “WMD”) that employs a wearable article to facilitate capture of signals representing patient parameters as the patient goes about his or her day and/or night before, during, and/or after treatment sessions. The wearable medical device processes the signals into comprehensive patient data that lends itself to interpretation related to the treatment. Patient data, for example, can reflect health status (e.g., physiological data), patient state, patient actions, and/or environmental conditions that may impact treatment. The patient data are transferred to analytical component(s) to determine proper courses of action regarding treatment of the patient, such as adjusting a time factor for treatments and/or varying types of treatments. The patient data may also be provided to devices of one or more healthcare professionals/providers. In some implementations, changes to a treatment plan may be made and rolled out in real time on the fly in response to a current or recently occurring patient event.

In some cases, time factors that can be adjusted in view of the patient data may include, for example, a time of day for treatment session(s), duration of the treatment session(s), how often to perform the treatment session(s), the period of time for all treatments to be performed and completed, repetition of previously performed treatment session(s), and/or discontinuation of one, some, or all treatment sessions.

Health issues that may be addressed by vagus nerve stimulation managed by the present management system may include one or more conditions, diseases, syndromes, illnesses, symptoms, or combinations thereof. For example, VNS can help POTS, Long-Covid, chronic fatigue, and other autonomic nervous system disfunction. Other health problems addressable by VNS using the present management system include epilepsy, heart failure, reactive airway diseases, gastric problems, depression, mood disorders, obesity, migraines, post-stroke motor rehabilitation, and a myriad of other health troubles. Some symptoms that may be treatment by vagus nerve stimulation using the present management system also include sensations of pain, emotions, ability to concentrate, occurrence of seizures, disorder of the limbic system.

In some implementations, a treatment plan may be revised according to patient data analysis described herein, to prescribe alternative treatment(s), different treatment modes (such as changing from a tVNS stimulation device to an implantable VNS stimulation device) or additional treatment(s). The management system may be configured to control such other treatments and/or monitor for patient data related to such other treatments. For example, with regard to POTS treatment, certain exercise training may expand blood volume and plasma volume, as well as increase cardiac muscle mass and heart size of a patient. These impacts in turn have been associated with improvement in symptoms, and therefore exercise training as supplemental treatment can be added to a treatment plan that includes tVNS. Patient data collected may reflect patient parameters that include patient state during exercise, exercise intensity, patient compliance, and combined effect of such exercise in interpreting the tVNS results. Further example supplemental treatment may include medications that may augment VNS treatment and be monitored (e.g., by analyzing patient data for detected patient parameters and/or information on prescribed medications from a healthcare provider) by the present management system in combination with stimulation treatment sessions.

In some implementations, the WMD may also provide cardiac pacing components for detected bradycardia, asystole, and to be used in POTS treatment of symptoms. In some implementations, the WMD may be configured to provide alternative or supplemental treatment (in addition to or instead of the described VNS treatments) in the form of cardiac pacing therapy, such as the WMD employing heart pacing components.

It is understood that POTS episodes can be triggered when a patient changes positions (for example, standing up from a sitting position). In some cases, cardiac pacing therapy may increase blood pressure of the patient prior to such position changes, which may facilitate lessening (or prevention) of relative jumps in heart rate and address potential fainting of the patient upon changing positions. The pacing may, for example, be initiated via a user interface. (e.g., the alert button on the WMD, a voice command functionality, a button or actuator on the stimulation device it, etc.). The temporary period of pacing therapy may be initiated prior to changing positions. In some implementations, the pacing therapy may be initiated upon the WMD sensing the patient changes positions from lying or sitting positions to standing with anticipation that POTS symptoms may otherwise be triggered. The control unit of the WMD may; further be configured to control (e.g., initiate, turn off, regulate electrical output levels, frequency of pulses, etc.) the pacing components of the WMD for pacing treatment sessions or an external cardiac pacing device. The pacing treatment session duration may be preset (e.g., lasting 1-5 minutes, or less than 1 minute as needed to achieve the intended benefits). In some implementations, pacing session duration may be controlled by a motion detector sensor that detects when the patient has completed the change of position. Such pacing treatment may be included in the treatment plan for the patient.

Patient data may represent patient physiology, state, occurrences, actions, environment regarding the pacing treatment. Revisions to the treatment plan based on analysis of the patient data may; include changes to pacing treatment, such as changes to timing factors, modification of treatment parameters, adding pacing sessions, or discontinuing pacing sessions.

Greater detailed data about the patient's day to day status enables personalized treatment decisions, such as how often to treat and duration, time of day of the treatment, whether the treatment needs to be reinstated once the regime is complete, whether the intended response has been achieved and sustained, whether a different form of stimulation device, such as implantable stimulation device, or different treatment area of the body is necessary, etc. Data may be reviewed to assess patient compliance of treatment requirements. For example, changes to the treatment plan may be warranted to elicit better patient compliance, such as time of day for treatment, duration, accommodating the patient's daily routine or special activities. The system may delay treatment during certain patient actions, such as the patient driving. Such changes may be logged and provided to the healthcare professional via healthcare provider devices for consideration in evaluating treatment results.

Use of the terms “treatment”, “treat”, and variations thereof, refer to attempts at curing the health issue, causing remission of the health issue, ameliorating or lessening at least some of the symptoms of the health issue, or otherwise benefiting the patient in experiencing the health issue.

The term, “patient data” refers to data that are generated from signals that are generated by sensors (also referred to as signal source components) that sense, retrieve, observed, determined, and/or converted from signal source components, e.g., sensors or transducers. The patient data represents patient parameter(s) and may include indicators of patient parameters that may be expressed in quantitative and/or qualitative terms. In some implementations, the patient data may be raw measurements that can be further processed during analysis by a patient communication device and/or remote data management device. Analysis may be used to spot trends (such as medication intake correlated with stimulation sessions), patterns, and other indicators of important information. In some implementations the analysis may include determinations of whether or not a threshold is crossed. The term “meeting” a threshold as used in this description means a value being equal to or exceeding the threshold.

The WMD employs a wearable article (also referred to as a “support structure”) and is configured for continuous long term wear by a patient, of at least fourteen (14) days, such as 2 months. Some implementations of the WMD may be worn for a few months or longer, including one or more years. The WMD can be worn during treatment by a stimulation device, during a preparation pre-treatment period of time prior to treatment by the stimulation device and/or during a post-treatment monitoring period of time after treatment by the stimulation device is completed. The term, “continuous” wear of the WDM is understood to include daytime use, which may be from many hours of the day and fulltime at night, to full daytime hours and full nighttime use. In some implementations, the WDM may be worn without stop except for temporary daytime removal during brief activities that may expose the management system to potentially adverse conditions, such as water contact, e.g., bathing or swimming, cleaning of the wearable article, repair or maintenance of the WDM, etc. Thus, “continuous use” is intended to include such brief periods of non-use.

Prior VNS treatment devices may be limited in monitoring narrow results of a stimulation and may modulate characteristics of signals such as voltage, current, frequency, waveform of signal. The present management system includes a wearable device with sensors for long-term and continuous sensing. For example, the present wearable article may be a garment, such as a vest, worn under regular clothes. The present management system enables detection and analysis of day to day occurrences of a patient, such as patient state, actions, environment, that can significantly impact treatment results and warrant changes to treatment plans. With the present management system, combinations or vast amounts of data collected over the course of a day (e.g., 20-24 hours a day) may be analyzed in a comprehensive manner to identify trends and impacts of various combinations of treatments (including VNS stimulation sessions, medications, exercise), daily patient occurrences/patient states and actions, system information, etc., monitored along a time course to make knowledgeable changes to time factors and other characteristics of a treatment plan.

Some health issues treated by vagus nerve stimulation include various symptoms that cross over several medical disciplines. Often, healthcare professions are restricted in obtaining comprehensive real time information on a patient. For example, various specialized healthcare professionals work with POTS patients including cardiologists, neurologists, pediatricians, primary care physicians, etc. In some cases, a team of experts may be enrolled to provide care. The present management system facilitates establishing a communication link with the data management device that can share patient data and/or revised treatment plans across remotely located providers. Reports from the data management system may be generated and transmitted to a healthcare provider so that a physician monitoring the progress of patient is informed about a condition that is improving, not improving, or deteriorating. Various healthcare providers may further input patient information, such as medications prescribed by the provider, into the data management system to be used in analysis for treatment plans.

The present management system provides additional benefits and avoids prior limitations, which will be apparent by this description. The medical management system is not limited to the described use cases below. As can be recognized by the description, there are numerous other situations in which the medical management system may be employed, with various sensors and/or transducers to gather and analyze a variety of patient data to determine treatment plans.

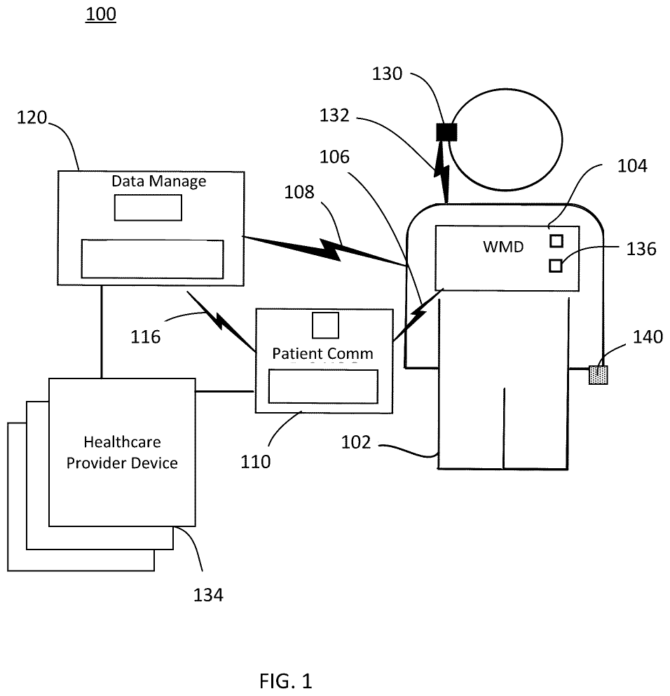

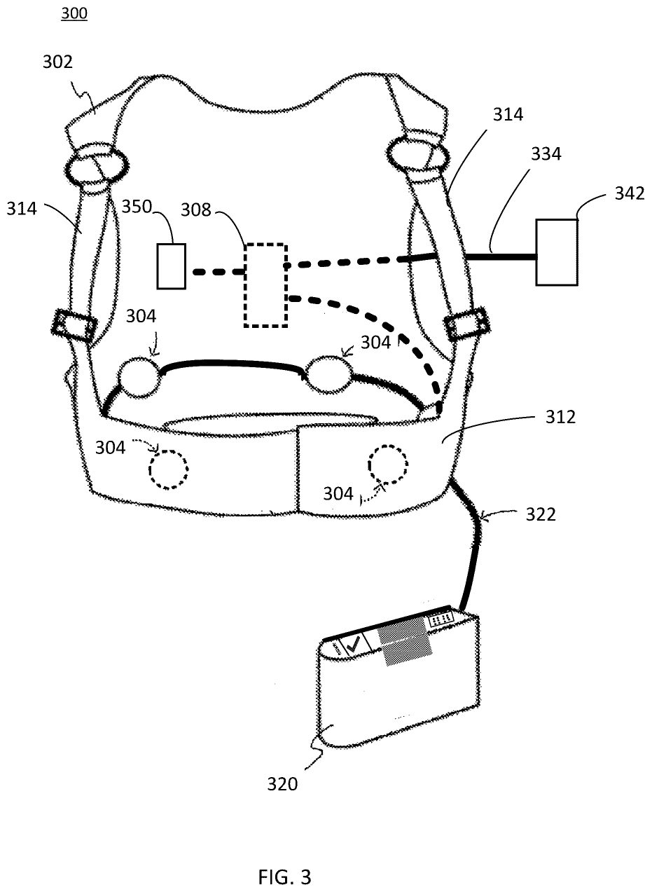

FIG. 1 shows an overview of an example of the medical management system 100 that employs a WMD 104 including a wearable article (described below with regards to FIG. 3) worn by a patient 102, such as an ambulatory patient, to generate patient data, a data management device 120 to analyze the patient data to generate a treatment plan and/or revisions of treatment plans, and a stimulation device 130 to generate electrical pulses to provide tVNS. Implementations of the management system 100 can also include a patient communication device 110 that can serve as an intermediary device to transfer patient data to a data management device 120. In still some implementations, one or more healthcare provider devices 134 may receive patient data and may also transmit recommendation information to the data management device 120 for treatment plans and/or revisions.

The WMD 104 includes one or a plurality of various sensors 136. For illustration purposes in FIG. 1, the sensors 136 are shown as positioned internal (in or on the wearable article). The sensors 136 may also include external sensors not on or in the wearable article. Sensors 136 represent one or multiple sensors, internal and/or external positioned. In some implementations, the sensors 136 can be construed as encompassing more than one individual sensor and various types of sensors 136 that detect a variety of patient parameters. In some implementations, a patient 102 or representative of the patient may provide prior consent to the medical management system 100 detecting, storing, transmitting, and analyzing particular patient parameters associated with the patient.

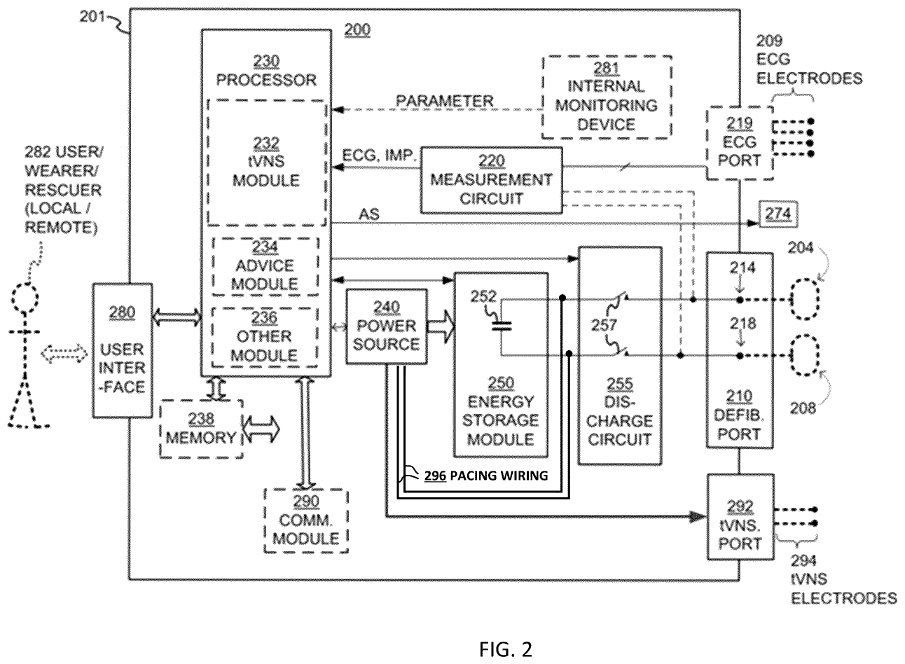

At least some of the sensors 136 are in contact with, or in close association to the patient 102 to detect patient parameters. The sensors 136 detect aspects of patient parameters and translate the information into electrical signals such that the signals reflect the patient parameters. For example, some sensors may include one or more electrodes may contact the body of the patient and conduct electricity. The signals are converted into patient data by a control unit of the WMD 104. Details of an example WMD 104 are shown in FIG. 2 described below.

In some implementations, the wearable article may include and/or accommodate different types of internal sensors 136. Sensors 136 may be integrated with the material of the wearable article. Other sensors 136 may be removeably attached to the wearable article. In such implementations, the wearable article may accommodate the sensor via an attachment structure, such as a receptacle, strap, fastener, etc. that is adapted for a particular type of sensor. In addition, some implementations of the WMD may be in communication with sensors external to (e.g., not in contact with) the WMD. For example, one or more sensors may be coupled to or integrated with the stimulation device 130 and in communication with the WMD for the WMD to receive signals.

Biological sensors may produce signals in the form of electrocardiogram (ECG) from electrodes, peripheral capillary oxygen saturation (spO2) from an oximeter, temperature from a thermometer, and so on. Biological sensors detect patient parameters such as patient physiological parameters including heart rate, breathing characteristics, blood oxygenation, body temperature. Patient state parameters detected by various sensors 136 may include body position, body orientation, stress level (such as a combination of heart rate variability, skin/body temperature, skin sweat, voice patterns, gestures, etc.), consciousness, a seizure (such as pulse, falling, muscle tone, sudden movements like twitching, grunting noise, etc.), falling by an accelerometer, and so on. Some example sensors for POTS symptoms that may be employed by the present management system are describe in International Patent Publication WO 2018/048825, “System to Diagnose and Manage Orthostatic Intolerance,” filed Sep. 6, 2017, the contents of which is incorporated herein by reference.

Some biological sensors employed may be configured to detect particular patient parameters that indicate patient compliance with supplemental treatments, such as taking medication. Such particular patient parameters may be processed into patient data that is analyzed by comparing the patient data to predefined model parameters expected when the patient takes the proper dosage of medication at the prescribed time according to the treatment plan. Variation of the detected patient parameters form the expected model parameters may indicate that a patient failed to comply with the treatment plan. Further analysis may provide insight on the biological effects of the medication with the stimulation treatment results. Such insight may be used by the data processing system to revise a treatment plan as needed, such as vary medication and/or stimulation treatment sessions.

A patient sleep state may be indicated by a combination of sensor signals. The management device may output a treatment plan or revisions according to a patient sleep determination. For example, insomnia or sleep disruptions may suggest lack of effectiveness of a treatment and other types of treatment may be ordered. In some implementations, a timing factor for treatment sessions may be changed to be conducted or avoided during a sleep state. For example, the management system detecting sleep on the fly may and result in a revision of the treatment accordingly. The system determining a sleeping pattern may result in the system revising a time of day schedule for treatment sessions.

Sensor employed to detect sleep may include an environmental light sensor to indicate the ambience of the environment since most patients sleep in darker environments that can indicate a sleep state. A clock may also indicate the time of day and have an accumulated history indicating what time of day the patient is normally asleep. The respiration rate and pattern may also be used, as well as a sound sensor that can indicate a sleep sound of the patient, such as snoring, for example. In some implementations torso position/orientation may be detected, such as by a 3-D accelerometer. Certain torso position and/or orientation may correlate with symptoms of the health issue and/or impact on treatment effectiveness for particular health issues. For example, sleep apnea is less frequently found in patients who sleep in a lateral decubitus (on side) position and more common or pronounced in a supine position without elevation. Each of these sensors may accumulate a history to help the management device 120 to accurately detect that the patient is in a sleep state.

Some environmental sensors may detect various aspects of the patient environment such as high or low environmental temperatures, a global positioning system (GPS) to detect patient location, a speed can be detected as a rate of change of location over time, a moisture detector to detect wetness level that may interfere with electrical stimulation treatments, a microphone to detect environmental noises, e.g., noise levels, that may trigger physiological effects in the patient, such as noise triggered stimulation overload in a patient. Other environmental sensors can be employed to detect aspects that can impact patient physiology and/or treatment of the patient.

In still some implementations, patient data may be generated by a patient predictive artificial intelligence (AI) model that outputs predictions of patient parameters, rather or in addition to processing signals from sensor detection. For example, the patient predictive AI model may use as input, patient data generated from sensors detecting patient state and actions. The AI model may output predicted actions by the patient, such as future movements by the patient. The patient predictive AI model may be trained on patient routine information that shows trends of the patient extracted by patient data. Training datasets may also include a sample of typical actions commonly performed by patients when certain patient data is detected.

This description of types of sensors and patient parameters is not exhaustive and additional types of sensors and patient parameters that can be detected and related to treatments or various health issues may be employed. In some implementations, supplemental patient information may also be retrieved and provided to the data management system, such as medical history of the patient, patient demographics, event history, and so on.

In some implementations, treatment data is also captured by the WMD 104 that can be synced in time with the patient data to provide a full picture of the patient treatment and response. A report of the patient data synced with the treatment data may be generated by the WMD and/or data management device. The treatment data may be used to confirm treatment sessions are conducted as described in a treatment plan. Delivery characteristics, such as time of the actual treatment, point of delivery area of the body in contact with the stimulation device, any anomalies in the delivery of treatment (such as a weak point of contact or slippage of contact), etc. may be extracted from the treatment data. Delivery characteristics may be used by the system to determine whether the patient complied with the treatments as required by treatment plan. The treatment data may also represent additional operations of the stimulation device such as signal features (frequency, waveform, etc.) to further determine whether stimulation treatment sessions had been administered by the stimulation device according to the treatment plan. Any variability or malfunction of the stimulation device may be detected. A revised treatment plan may require a correction of signal features of the stimulation device, including replacement or repair of the stimulation device, change in electric pulses, or repositioning of the stimulation device.

In some implementations, alternative or supplemental treatments, such as specified exercise or diet may be included in the treatment plan. The treatment data may be extracted from patient data obtained by sensors that indicate whether the patient complied with such alternative or supplemental treatments.

In some implementations, the WMD 104 can include treatment components such as an integrated stimulation device 130 and/or other types of treatment components that can be employed as a supplement treatment along with VNS sessions or instead of VNS treatment. In some implementations, the WMD can be in the form of a wearable cardioverter defibrillator (WCD) and can provide tVNS pulses using an internal capacitor, similar to the system described in U.S. patent application Ser. No. 18/446,391 (hereinafter '391) entitled “WCD Pacing Pulse Generation” filed Aug. 8, 2023, which is incorporated by reference herein in its entirety for all purposes. Instead of or in addition to generating pacing pulses for the heart, the system of the '391 may be modified to provide tVNS pulses to a tVNS port (as described below with regards to FIG. 2). Otherwise, these implementations are substantially similar to the implementations described herein. An example of an external stimulation device 130 for tVNS treatment can include gammaCore device (by electroCore, Rockaway, NJ).

Whether the stimulation device 130 is integrated with the WMD 104 or external, the stimulation device 130 is at least partially controlled by the WMD 104, for example, the stimulation device 130 may be paired with the WCD 104. In some implementations, additional controls of the stimulation device 130 may include user input, healthcare professional input, preprogrammed on-board controls of the stimulation device, and other control interface.

In some implementations, a power unit 140, such as a portable charging case or charging pad, may be included in the management system 100 to recharge a battery of the stimulation device 130 when not in use by the patient. The stimulation device may be configured to be recharged by contacting or being inserted into the power unit. For example, the stimulation device may include a receiver to accept an electromagnetic field of the power unit 140 and convert into electrical current. The power unit 140 may be wireless, such as stored power that is replenished by contacting a pad, or wired using a cable to a power source.

The stimulation device 130 may be configured to be positioned external to and in contact with a portion of the body of the patient when in use. For example, the stimulation device 130 may be configured to be removably coupled to the auricle region of the patient, such as the tragus or conchal region. In some implementations, the stimulation device may be partially and removably inserted into a piercing of the ear similar to an earring, such as through the tragus in which electrodes may be inserted into the piercing.

In some implementations, the stimulation device 130 may be multi-function to provide VNS treatment and also provide recreational and/or additional health components. For example, the multi-function stimulation device 130 may include components to provide additional treatments for the same health illness or other health illness. The stimulation device 130 may also be configured to resemble and/or provide similar auditory functions as an earbud or headphone. Other formats of the stimulation device may include a multi-function hearing aid (that provides hearing assistance as well as tVNS treatment) or resemble a hearing aid that wraps around the ear with a housing located behind the ear, embedded in or attached to the arms (temples) of eyeglasses or sunglasses, and other formats.

In some implementations, the stimulation device 130 may communicate with and receive audio signals from the patient communication device 110 and output audio to the patient 102 via speakers of the stimulation device 130. In this manner, the stimulation device may output, for example, music, phone calls, video sounds during stimulation treatment or when stimulation treatment is not being performed. The multi-function stimulation device may further couple to the control unit of the WMD 104 and/or patient communication device 110 to receive treatment related information and output audio information accordingly, such as prompts or alerts to the patient 102 or caregiver of the patient.

In still some implementations, the stimulation device may be configured to activate and/or deactivate stimulation treatment via various triggers that may be consistent with the treatment plan or vary from the treatment plan as desired. For example, the stimulation device may include one or an array of microphones that detect the user voice command to activate stimulation, deactivate stimulation, change a system parameter for stimulation output, etc.

In still some implementations, the stimulation device 130 can also provide stimulation of the vagus nerve via bone conduction of sound or vibration generated by the device. Such stimulation device may be worn by the patient proximate to the skull of the patient near the ear, similar to bone conduction hearing aids. See, for example, Howland R. H. Vagus Nerve Stimulation. Curr. Behav. Neurosci. Rep. 2014; 1:64-73, the contents of which is incorporated herein by reference.

The stimulation device 130 may also be implanted or partially implanted in the body. A portion of a semi-implantable stimulation device that is installed outside of the body may include a receiver to receive control signals from the WMD. In some implementations, tVNS therapy can be delivered to other parts of the external body of the patient such as the neck for the cervical branch vagus nerve.

In the example management system 100 shown in FIG. 1, the stimulation device 130 is external to the WMD 104 in communication with the WMD 104 by a receiver/transmitter of the stimulation device via communication link 132. For example the communication link 132 may be used for the WMD 104 to transmit control information to the stimulation device 130. The communication link 132 may also be used for the stimulation device 130 to transfer operations information to be included in treatment data to reflect administration of treatments.

Communication link 132 may include various wired connections, or wireless short range communication technologies, such as Bluetooth, Bluetooth Low Energy (BLE), Zigbee, WiFi, Z-Wave, Ultra-Wideband, Near-Field Communication, etc. The stimulation device may be integrated with and a component of the wearable device, may be external and in communication with the wearable device, or both modalities by the stimulation device being partially integrated with the wearable device and partially external. The stimulation device may be transcutaneous, fully implantable in a patient or partially implantable in the patient in which some components are implanted internal in the body and some components remain external of the body. The stimulation device may include one or more stimulation devices or electrical component units to release electrical pulses to one or both vagus nerves.

Examples of other treatment components of a WMD may include components to provide defibrillation treatment and/or heart pacing as in a WCD, for example, as disclosed in the U.S. Pat. No. 8,838,235, titled: “Wearable Defibrillator System Communicating via Mobile Communication Device,” or the U.S. Pat. No. 10,449,370, titled: “Network-accessible data about patient with wearable cardiac defibrillator system,” both incorporated herein by reference for all purposes.

The WMD 104 transfers patient data, which can be in the form of stored data blocks or periodically captured data and transmitted in real-time, a real-time data stream, and the like. The data is transmitted to the data management device 120 via communication link 108. In some implementations, an example of the data management device 120 includes Kestra CareStation® remote data platform available from Kestra Medical Technologies, Kirkland WA. In some implementations, the patient data is transmitted from the WMD 104 to the patient communication device 110 (such as such a smartphone e.g., with a patient software application installed therein or smartwatch) via transfer link 106 to be further transmitted from the patient communication device 110 to the data management device 120 via communication link 116. Communication links 106, 108, 116, and/or 132 may employ a number of different near or distance communication technologies.

In some implementations, transfer of patient data may be pushed to the data management device 120 and/or devices of healthcare provider device 134 on a regularly scheduled basis, such as every 15 minutes. However, transfer of patient data may also be pulled by request of the data management device 120 and may be requested by a user. In some implementations, the data management device 120 may be implemented as a cloud service and functions may be distributed across one or more networked devices.

At the data management device 120, analysis of the patient data and treatment data are performed by a data analysis module. Analysis criteria may be stored in memory at the data management device and used as rules or guidelines in the data analysis. For example, the analysis may determine trend or pattern of the data or combination of different types of data to match with trend or pattern criteria stored in memory as representing a determination about the data, such as effectiveness of the treatment.

Analysis criteria may further include a particular situation or occurrence of the patient that is happening or recently happened. Such situational analysis may include comparing the patient data to stored situational information from prior monitoring of the patient or occurrences of sample other patients. Some examples of such analysis criteria may include the patient taking medication compared to expected patient data when taking such medication or when the subject patient experiences treatment sessions without the medication. Correlations between a drug (e.g., types or dosage), VNS stimulation treatment, and/or patient data (i.e., patient parameters) may be analyzed to determine appropriate treatment plans.

In some cases, treatment data may be analyzed to determine treatment compliance with a current treatment plan. The treatment data may include a reporting of the operations of the stimulation device, which is compared to expected operations for the stimulation device to follow the treatment plan. At times patient data coupled with treatment data may reflect compliance of the patient with the treatment plan. Treatment data may also reflect system parameters of a WMD and/or stimulation device, which can include system identification, battery status, system date and time, reports of self-testing, records of data entered, records of episodes and intervention, and so on.

In some implementations, weights may be attached to particular types of patient data and analysis criteria to factor in data or criteria that are more significant to a particular treatment, patient, and/or health issue. In some implementations, the data management device 120 may determine a level of a patient parameter, such as mild, moderate, and severe, for example, based on the management system tracking patient parameter events during a particular time period.

In some implementations, provider information can be received from professionals via healthcare provider device(s) 134 through communication links with the data management device 120 and integrated into a comprehensive treatment plan including revisions to existing treatment plans based on captured patient data. The data management device performs patient data analysis and may also integrate input from diverse healthcare providers.

In some implementations, one or more artificial intelligence (AI) models to assist in determining treatment plans, including revisions to existing treatment plans. Treatment plan AI models may use current patient data, health issue information, and current treatment plans (if any) as input data. Other input data may include historical stored patient data regarding past treatments, patient characteristics, etc. The AI model outputs treatment plan information that would likely benefit the patient based, at least in part, on the input data.

Treatment planning AI model(s) may be trained with datasets that may include sample information that mimics the input data. In some implementations, the techniques to train the AI model may employ supervised classification algorithms, such as logistic regression algorithms. In some implementations, unsupervised or semi-supervised techniques may be employed. Machine learning can also be employed to enable self-learning by analyzing training datasets and improve performance over time. The AI model conducts predictive analysis using the training dataset. The training of the AI model may include analyzing trends and patterns in various combinations of sample patient data and treatment data as related to various treatment plans that lead to positive predictive results. Based on the analysis, the AI model outputs a result of the analysis that predicts a treatment plan (including any revisions) for a particular sample situation. Where there is discrepancy information based on a training output result from prior AI model output results and may be used for retraining the AI model. For example, if a treatment plan revision is outputted by the AI model and found to not to have an intended result, such as failure to achieve a target therapeutic effect, failure to address patient compliance of a treatment plan, etc., the discrepancy information may be fed back into the AI model for retraining.

The communication links 106, 108, and/or 116 may include one or more WANs (Wide-Area Networks) and/or LANs (Local-Area Networks), which may be wired and/or wireless. In some examples, the communication links 106, 108, and/or 116 may include the Internet and/or one or more cellular networks, among other networks. For example, the communication links 108, and/or 116 may provide a connection, for example, through a local network to a host computer or to data equipment operated by an Internet Service Provider (ISP). The ISP in turn provides data communication services through the world wide packet data communication network (the “Internet”). In some implementations, communication links 106 (between the WMD 104 and patient communication device 110 and 132 (between the WMD 104 and stimulation device 130) may employ short range communication technology and/or wired communication technology, and communication links 108 (between the WMD 104 and data management device 120) and 116 (between the patient communication device 110 and data management device 120) may employ long-range communication technologies. Combinations of short and long range communication is also possible for any of the communication links.

The communication links 106, 108, 116 may operate according to one or more communication protocols, such as Bluetooth™, LTE (Long-Term Evolution), CDMA (Code Division Multiple Access), WiMax (Worldwide Interoperability for Microwave Access), WiFi (Wireless Fidelity), WiFi Direct (Wireless Fidelity Direct), EDGE (Enhanced Data rates for GSM (Global System Mobile) Evolution), 3G (Third Generation), 4G (Fourth Generation), 5G (Fifth Generation), HTTP (Hyper-Text Transfer Protocol), TCP (Transmission Control Protocol), SIP (Session Initiation Protocol), device contact based transfer protocols, device movement based pairing protocols, and other communication protocols.

It should be understood that the communication links 106, 108, 116 may include multiple, distinct networks that are themselves communicatively linked or a single network. The communication links 106, 108, 116 could take other forms as well.

In some implementations, the WMD 104 may instead or also transfer health data, such as data blocks and/or a health data stream, directly to data management device 120 via communication link 108. As the data management device 120 may be positioned remote from the WMD 104, communication link 108 may employ a variety of communication technologies configured for distant communication, such as described above with regard to communication link 106. In some implementations, communication link 106 and communication link 108 are a same communication network rather than individual communication links.

The patient communication device 110, may be any computing device such as a mobile device, positioned local to the patient or to a person (e.g., local caregiver) in immediate contact with the patient during signal collection and treatment sessions, such as a local caregiver in the proximate area of the patient. The patient communication device 110 may facilitate offloading of tasks to save power and conserve resources from the WMD, that otherwise may be consumed by intensive data transfer processes.

In some implementations, the patient communication device 110 may receive supplemental information, such as patient and/or caregiver input via a user interface of the patient communication device 110. Such supplemental information may be transferred to the data management device 120 for analysis as additional patient data. Supplemental information may be objective or subjective about the patient, for example symptoms, additional wearer feedback, etc. In some implementations, supplemental information includes information about patient actions, such as wear compliance, steps taken, etc.

The depictions in FIG. 1 is not to be construed as limiting components of the medical management system 100 and how the management system 100 is implemented. The medical management system 100 can be implemented in different ways with additional or less devices/components. For example, in some implementations, the functions of the patient communication device 110 may include the WMD 104 and the patient communication device 110 may not be needed. Additional features and combinations of features are possible.

FIG. 2 shows a top-level functional block diagram of pertinent hardware/software to generate tVNS pulses. In some implementations, the WMD 200 can be configured to provide cardiac treatments directly to the patient, such as defibrillating or cardiac pacing to the patient, in addition to managing VNS treatments. Defibrillation can be performed by defibrillate components of the WMD implemented using a WCD to deliver an electrical charge to the body of the patient in the form of an electric shock. The electric shock can be delivered in one or more pulses. In other implementations, the WMD may be a wearable monitoring device that monitors cardiac activity (e.g., the ASSURE® system, wearable ECG device, by Kestra Medical Technologies, Inc., Kirkland WA) or other patient physiological parameters.

The components shown in FIG. 2 can be provided in a housing 201, which may also be referred to as casing 201 of the WMD 200. The WMD may include various an internal monitoring device 281. WMD 200 may include a sensor port 219 in housing 201, which is also sometimes known as an ECG port. Sensor port 219 can be adapted for plugging in sensing electrodes 209, which are also known as ECG electrodes and ECG leads. Sensing electrodes 209 can be connected continuously to sensor port 219, instead. Sensing electrodes 209 (e.g., sensors) are types of transducers that can help sense an ECG signal, e.g., a 12-lead signal, or a signal from a different number of leads, especially if they make good electrical contact with the body of the patient and in particular with the skin of the patient. As with defibrillation electrodes 204, 208 described below, the wearable article can be configured to be worn by patient 282 so as to maintain sensing electrodes 209 on a body of patient 282. For example, sensing electrodes 209 can be attached to the inside of a wearable article for making good electrical contact with the patient, similarly with defibrillation electrodes 204, 208.

A measurement circuit 220 may be provided to senses one or more electrical physiological signals of the patient from sensor port 219 and/or obtain physiological signals through nodes 214, 218. For example, the patient parameter can be an ECG, which can be sensed as a voltage difference between electrodes 204, 208. In addition, the patient parameter can be an impedance, which can be sensed between electrodes 204, 208 and/or between the connections of sensor port 219 considered pairwise. Sensing the impedance can be useful for detecting, among other things, whether these electrodes 204, 208 and/or sensing electrodes 209 are not making good electrical contact with the patient's body. Measurement circuit 220 can then render or generate information about them as inputs, data, other signals, etc.

WMD 200 that includes a WCD for defibrillation treatment includes a defibrillation port 210, which can be a socket in housing 201. Defibrillation port 210 includes electrical nodes 214, 218. Leads of defibrillation electrodes 204, 208 can be plugged into defibrillation port 210, so as to make electrical contact with nodes 214, 218, respectively. It is also possible that defibrillation electrodes 204, 208 are connected continuously to defibrillation port 210, instead. Either way, defibrillation port 210 can be used for guiding, via electrodes, to the wearer at least some of the electrical charge that has been stored in an energy storage module 250. The electric charge may be used for defibrillation shock, pacing, and so on.

WMD 200 also includes a processor 230. Processor 230 may be implemented in a number of ways in various embodiments. Such ways include, by way of example and not of limitation, digital and/or analog processors such as microprocessors and Digital Signal Processors (DSPs), controllers such as microcontrollers, software running in a machine, programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 230 may include, or have access to, a non-transitory storage medium, such as memory 238 that is described more fully later in this document. Such a memory can have a non-volatile component for storage of machine-readable and machine-executable instructions. A set of such instructions can also be called a program. The instructions, which may also be referred to as “software,” generally provide functionality by performing acts, operations and/or methods as may be disclosed herein or understood by one skilled in the art in view of the disclosed embodiments. In some embodiments, and as a matter of convention used herein, instances of the software may be referred to as a “module” and by other similar terms. Generally, a module includes a set of the instructions so as to offer or fulfill a particular functionality. Embodiments of modules and the functionality delivered are not limited by the embodiments described in this document.

Processor 230 can be considered to have a number of modules. One such module can be a tVNS module to provide tVNS pulses as an integrated stimulation device. For example, in some implementations, the tVNS module 232 may cause power source 240 (e.g., a charger used to charge capacitor 252) to enter a mode to output tVNS pulses to tVNS port 292 and to the patient via tVNS electrodes 294 that are coupled to the tVNS port 292. In some implementations, the tVNS electrodes are implemented as an ear clip that can be attached to the patient's ear at the tragus. In some embodiments, the tVNS pulses are generated at 20 Hz, with pulse width 200 μs, with a current ranging between 6 mA to 25 mA, and delivered to the patient periodically, such as daily for a predefined duration, such as 1 hour. In some embodiments, the tVNS pulses are provided to treat POTS, similar to the treatment described in Stavrakis, Noninvasive Vagus Nerve Stimulation in Postural Tachycardia Syndrome, J Am Coll Cardio: Clinical Electrophysiology, 2023, which is incorporated herein in its entirety for all purposes.

In addition, the patient physiological parameters monitored by sensors of the WMD 200 can be used to interpret effectiveness of the tVNS treatment, generate treatment plan revisions, such as adjust the parameters of the tVNS treatment, and/or determine whether the patient needs an implanted VNS device. For example, the patient's sensed ECG from measurement circuit 220 and values of other signals (e.g., from internal monitoring device 281), may be used by the tVNS module 232 to capture patient physiological data prior to tVNS sessions, while tVNS treatment is being provided, and/or during a post-treatment period.

Another such module in processor 230 can be an advice module 234, which generates advice for the patient and/or caretaker on actions to be taken with regards to the treatment plan and/or stimulation sessions. For example, the advice module 234 may output a notification to the patient that a VNS treatment session is about to commence and/or ending. In some implementations, the notification may further include advice such as to lie sit or down, not eat, etc., during the treatment. The notifications may be outputted via the user interface 280 and may take the form of audio through a speaker, visual display, tactile indications (such as vibration), etc. In some implementations, notifications may be outputted via an interface at the patient communication device, such as 110 in FIG. 1. For example, a notification may be generated by the advice module 234 and to the patient communication device for display, sound output, tactile indicator or other outputs. Other modules 236 are possible.

WMD 200 optionally further includes a memory 238, which can work together with processor 230. Memory 238 may be implemented in a number of ways. Such ways include, by way of example and not of limitation, volatile memories, Nonvolatile Memories (NVM), Read-Only Memories (ROM), Random Access Memories (RAM), magnetic disk storage media, optical storage media, smart cards, flash memory devices, any combination of these, and so on. Memory 238 is thus a non-transitory storage medium. Memory 238, if provided, can include programs for processor 230, which processor 230 may be able to read and execute. More particularly, the programs can include sets of instructions in the form of code, which processor 230 may be able to execute upon reading. The programs may also include other information such as configuration data, profiles, scheduling etc. that can be acted on by the instructions. Executing is performed by physical manipulations of physical quantities, and may result in functions, operations, processes, acts, actions and/or methods to be performed, and/or the processor to cause other devices or components or blocks to perform such functions, operations, processes, acts, actions and/or methods. The programs can be operational for the inherent needs of processor 230, and can also include protocols and ways that decisions can be made by advice module 234.

In addition, memory 238 can store prompts for user 282 if this user is a local rescuer. Moreover, memory 238 can store data. This data can include patient data (such as patient physiological data, patient state data, environmental data, etc.), and treatment data, for example as learned by internal monitoring device 281 and the aforementioned optional outside monitoring device. The data can be stored in memory 238 before it is transmitted out of WMD 200, or be stored there after it is received by WMD 200.

WMD 200 can optionally include a communication module 290, for establishing one or more wired or wireless communication links with other devices, for example, as described above with regard to FIG. 1. Communication module 290 may also include such interconnected sub-components as may be deemed necessary by a person skilled in the art, for example an antenna, portions of a processor, supporting electronics, outlet for a telephone or a network cable, etc.

The communication module 290 may include software that enables communications of the user interface 280 over a network such as the HTTP, TCP/IP, RTP/RTSP, protocols, wireless application protocol (WAP), IEEE 902.11 protocols, and the like. In addition to and/or alternatively, other communications software and transfer protocols may also be used, for example IPX, UDP or the like. The communication network may include a local area network, a wide area network, a wireless network, an Intranet, the Internet, a private network, a public network, a switched network, or any other suitable communication network, such as for example cloud networks. The network may include many interconnected computer systems and any suitable communication links such as hardwire links, optical links, satellite or other wireless communications links such as Bluetooth, Wi-Fi, wave propagation links, or any other suitable mechanisms for communication of information. For example, the network may communicate to one or more mobile wireless devices, such as mobile phones, tablets, and the like, via a base station such as a wireless transceiver.

In some implementations, communication module 290 may transmit wirelessly, on a daily basis, patient data over a network, such as the internet, for instance as described in U.S. Publication No. 2014/0043149, filed Aug. 6, 2013. This patient data can be analyzed automatically by algorithms of the data management device designed to determine treatment plans/revisions.

User 282 may include a person and/or computing system of a person or entity. For local interaction with the medical management system 200, the user 282 may include the patient or a local bystander. In some implementations, the user 282 may be a remote entity, such as a computing device of a remote person or a medical server device. For example, the user 282 may be a health support entity such as a doctor, caregiver, other health care provider, health care service, dispatch, technical service, an authorized person, and so on, including combinations thereof. The user may also include a medical server device or devices such as a cloud service, serving as a repository for health data of the patient.

A user interface 280 may transmit information to the user 282 and in some implementations may receive information from the user 282. The information may be pushed to user 282 in the form of a warning, in response to detecting a problem with the treatment, notification of an impending treatment session, or ending of such a session.

WMD 200 may also include a power source 240. To enable portability of WMD 200, power source 240 typically includes a battery. power source 240 is controlled and/or monitored by processor 230. Power source 240 may be controlled and/or monitored by processor 230.

WMD 200 may additionally include an energy storage module 250. Energy storage module 250 may temporarily store electrical energy in the form of an electrical charge, for example in preparation of administering a shock to the patient. In some implementations, energy storage module 250 can be charged from power source 240 to a predefined amount of energy, as controlled by processor 230. Energy storage module 250 may include a capacitor 252, which can be a single capacitor or a system of capacitors, and so on. In some implementations, energy storage module 250 may include a device that exhibits high power density, such as an ultracapacitor. Processor 230 may be configured to control discharge circuit 255 to discharge through the patient at least some of all of the electrical charge stored in energy storage module 250 to nodes 214, 218, and from there to defibrillation electrodes 204, 208, so as to cause a shock to be delivered to the patient. Discharge circuit 255 can include one or more switches 257, such as by an H-bridge, and so on. Discharge circuit 255 could also be thus controlled via processor 230, and/or user interface 280.

For pacer implementations, such as to provide POTS cardiac pacing therapy, an electrical pulse output may include employing at least some of the stored electrical charge through pacing wiring 296 that is caused, by processor 230 to be discharged via at least two of the therapy electrodes 204, 208 through the ambulatory patient 282, so as to deliver to the ambulatory patient 282 a pacing sequence of pacing pulses. The pacing wiring 296 is shown as two wires that bypass the energy storage module 250. As such, the energy for the pacing is provided by the power source 240 either directly via the pacing wiring 296, or through the energy storage module 250. And, in some implementations where only a pacer is provided, the energy storage module 250 may not be needed if enough pacing current can be provided from the power source 240.

The pacing pulses may be periodic, and thus define a pacing period and the pacing rate. There is no requirement, however, that the pacing pulses be exactly periodic. A pacing pulse can have an energy suitable for its purpose, such as at most 10 J, 5 J, usually about 2 J, and so on. In some implementations, the pacing pulse can have a constant current such as 100 mA, 150 mA, 200 mA, etc. The pulse has a waveform suitable for the intended purpose described in the treatment plan.

In some implementations, a fluid deploying mechanism 274 can be configured to cause at least some of the fluid to be released from the reservoir and be deployed near one or both of the patient locations to which electrodes 204, 208 are configured to be attached to the patient. The fluid deploying mechanism 274 may be activated prior to the electrical discharge responsive to receiving an activation signal (AS) from the processor 230.

WMD 200 may be used without the defibrillation functionality. For example, the defibrillation circuitry omitted (e.g., energy storage device 250, discharge circuit 255, defibrillation port 210. Such implementations include the patient physiological parameter monitoring circuitry and programming to capture this patient data so that the effectiveness, delivery parameters of the tVNS therapy, and/or whether the patient needs an implanted VNS device can be determined from the monitored patient physiological parameters, as described herein.

The WMD 200 can optionally include other components. In some implementations, one or more of system components may be customized for the patient. This customization may include a number of aspects, for instance particular treatment components may be integrated with the WMD 200 to treat and/or monitor patient parameters for certain health issues of the patient. A wearable article (e.g., 302 in FIG. 3) of the WMD 200 can be fitted to the body size, shape, or particular characteristics, such as wheelchair bound, of the patient.

In some implementations, customization may be based on detected parameters of the patent. For example, baseline physiological parameters of patient can be measured, such as the heart rate of patient while resting, while walking, motion detector outputs while walking, etc. Such baseline physiological parameters can be used to customize the medical management system, in order to make its treatment plan more accurate. Such parameters can be stored in a memory 238.

FIG. 3 shows a WMD 300 employing a garment type wearable article 302 configured to be worn on the torso of a patient. Torso-fitted wearable articles may reduce risk of damage that can occur with monitoring device attached to a limb of a person, such as the wrist or ankle, especially for patients with an active lifestyle. The WMD 300 may also include a control unit 320 and various circuitry 322, 334 such as cables for communication between components. The WMD 300 is shown to illustrate concepts about the management system and is not to be construed as limiting how the WMD 300 is implemented, or how the wearable article 302 is worn.

The wearable article 302 can be implemented in many different ways to provide a structural platform for particular components of the management system, such as sensors 304 that generate signals representing various patient parameters associated with the patient and a hub 308 that may collect and/or process signals from the sensors. Components may be detachably held by various pockets, receptacles, inserts, straps, removeable attachment mechanisms, e, g., hook and loop, etc.

The wearable article 302 may be a single structural element, or a combination of multiple structural elements. The example wearable article 302 shown in FIG. 3 is a vest garment style wearable article which is based on fabric material. The wearable article 302 includes a main body portion 312 configured to fit snugly around a torso of the patient 106 and two shoulder straps 314 with each shoulder strap configured to fit over each shoulder of the patient from the front side of the patient to the back side of the patient. In this manner, the wearable article 302 may encircle the patient without a need for adhesives to attach the wearable article onto the patient. The wearable article may also be implemented with a single shoulder strap, for example, that may wrap around the neck, or around one shoulder at an angle, or as a full vest rather than having shoulder straps. The main body portion 312 may be enclosed around the torso with fasteners, such as snaps, buttons, hook and loop, clasps, clamps, buckles, catchers, ties, etc., or with flexible fabric or elastic to allow stretch for slippage onto the torso. Other enclosures are possible.

The depiction in FIG. 3 is not to be construed as limiting how the wearable article 302 is implemented or worn. The wearable article 302 can be implemented in many different ways to engage with at least a portion of the torso of the patient and provide for long term continuous monitoring of the patient. For example, it can be implemented in a single element or a combination of multiple elements, which may be coupled together. In some implementations, wearable article 302 could include a vest, a half-vest, or other type of garment that engages with at least a portion of the torso. In some implementations, wearable article 302 could include a harness, one or more belts or straps, etc. that fit on a torso or other accessible parts of the patient. The wearable article 302 can also be worn around hips, over the shoulder, around appendages, etc. In implementations, such items can be worn similarly to analogous articles of clothing. Such items can be worn parallel to or underneath other articles of clothing.

In some implementations, the wearable article can be worn by being attached to the patient's body by adhesive material, for example as shown and described in U.S. Pat. No. 8,024,037. The wearable article 302 may also be implemented as described for the wearable article of U.S. Patent Application No. US2017/0056682, which is incorporated herein by reference. In such implementations, the person skilled in the art will recognize that additional components of the management system can be in a housing of a wearable article instead of being attached externally to the wearable article, for example as described in the US2017/0056682 document.

The wearable article 302 may include a sensors 350 to be used in collection of patient data. The sensor 350 may be positioned at various locations on or in the wearable article 302. Wearable article 302 may also be in communication with an external sensor 342 via communication link 334, in which external sensor 342 is not on or in the wearable article 302.

In implementations, the wearable article 302 can include one or more containers or housings, which can be waterproof. A person skilled in the art will recognize that in some implementations, additional components of the medical management system can be in a housing of the wearable article 302 instead of attached externally to the wearable article.

The wearable article 302 may be made of a variety of fabrics. In some implementations, material of the wearable article may also include conductive fabric for transportation of electric signal and/or power to and/or from components of the medical management system.

The wearable article may be removed from the patient, for example, prior to the patient engaging in brief activities and replaced onto the patient afterwards. In some implementations, the wearable article can be detachably secured to the patient by adhesive material. Various components, including electronic components including sensors, batteries, electrodes, and cables, may be detached from the wearable article, for example, to wash the wearable article, repair, or replace the wearable article.

Physiological parameters of the patient detected by the management system may include ECG, electrical impedance, DC current signals, respirational characteristics, blood oxygen level, blood flow, blood pressure, blood perfusion, pulsatile change in light transmission or reflection properties of perfused tissue, heart sounds, heart wall motion, breathing sounds, pulse, etc. In some implementations, a particular type of sensor may serve multiple roles in producing signals used to generate different types of health data. For example, a sensing electrode may provide both ECG signals and respiratory signals.

Patient state parameters detected by the management system may be used to determine sleep status of the patient. Patient state parameters may include recorded aspects of patient, such as respiration, motion, posture, whether they have spoken recently, what they said, and so on. In some implementations, the management system may compare currently detected patient state parameters with the patient history of these parameters to determine a trend or pattern of parameters that indicate a sleep state.

Environmental parameters detected by the management system can include ambient temperature and pressure. Moreover, a humidity sensor may provide information as to whether it is likely raining. Presumed patient location could also be considered an environmental parameter.

In some implementations, system parameters of management system can also be detected, including system time characteristics, such as date and time of day. Other system parameters may include a system identification, battery status, reports of self-testing, records of data entered, episodes, and intervention stored in memory of the management system, and so on.

In some implementations, the functions of control unit 320 may be entirely performed by a single unit or particular functions represented by unit 320 may be shared between two or more units, such as a hub 308. For example, the hub 308 may be housed in the wearable article 302 and may be in direct communication with sensors, such as internal sensors 350, sensing electrodes 304, and external sensors 342, to collect signals generated by the sensors. The hub 308 may also transfer the signals or patient data associated with the signals to the control unit 104.

Additional wearable article sensors 350 that may be housed by the wearable article 302 may include an audio detector such as heart-sound/phonocardiogram, patient temperature thermometer, GPS, pressure sensors, optical sensors such as photoplethysmography, etc. to produce signals used in generating health data about the patient. Such wearable article sensors 350 may be wired or wirelessly coupled to hub 308 to provide signals produced by the wearable article sensors 350 according to the sensors sensing of parameters of the patient.

In some implementations, the accelerometer may be readily adapted for use with the present teachings by those skilled in the art, as discussed in terms of a 3-axis accelerometer more fully in U.S. Pat. No. 11,083,906, filed Jan. 5, 2018, the contents of which are incorporated into this disclosure by reference. In some implementations, some features of the accelerometer may be used as described in U.S. Pat. No. 11,344,718, the contents of which are incorporated into this disclosure by reference. Other accelerometer devices and techniques may be employed to determine breathing characteristics, such as described in U.S. Pat. No. 10,159,421.

An audio detector may be provided to produce signals to represent sounds of the body of the patient, e.g., heart sounds, breathing sounds, and/or sounds of the surrounding area of the patient. The audio detector may sense heart activity, blood flow, snoring, or the patient talking. Placement of the audio detector on the wearable article provides for targeted detection. For example, a microphone may be positioned to detect frequency, volume, intensity, regularity, etc. of heart beat. The microphone may also detect frequency, volume, regularity, etc. of breaths.

Some examples of audio sensors that may be employed in some implementations are described in U.S. Pat. No. 11,058,884, the contents of which is incorporated by reference. Breath audio data may include frequency, volume, regularity, etc. of breaths. A frequency that is above a frequency threshold may indicate shortness of breath. Snoring may also be detected as sleep data to indicate the patient is asleep.

Some patient data may be combined with other patient data to for the data management device to determine a patient state and other analysis results.

External sensor 342 may be attached to other parts of the patient, for example, which may be more conducive to sensing a physiological parameter rather than at the torso. An example external sensor 342 may include an oximeter (also referred to as an “SpO2 sensor”) engaged with a body part, such as a finger, of the patient in which blood flow is easily detected. The oximeter may detect a reduction in blood oxygen level and signals or health data from the oximeter can be time-synchronized with other health data, such as the ECG and respiration data. Reduced blood oxygen saturation level may indicate a respiratory disturbance of the patient.

Another example of an external sensor 342 may include an environmental audio detector, such as a microphone, to detect environmental sounds, such as snoring, talking, noise in the surrounding area of the patient, etc. Environmental characteristic sensed by the environmental sensor may provide signals relevant to the state of the patient, such as whether the patient driving or exercising.

A data handling unit may process the signals at the hub 308 and/or control unit 320. The control unit 320 and/or hub 308 may process data, store data, use data for various determinations, and/or output patient data to data management device. In other implementations, a control unit 320 is in direct communication with sensors without use of a hub to collect health signals.

The control unit 320 of the medical management system is configured to perform various device controlling processes for operations of the management system, such as controlling stimulation device according to a treatment plan, receiving patient data, storing various data and information, implementing alternative treatments, such as cardiac pacing, performing operations on patient data, determining potential and/or actual medical disorders, receiving and implementing treatment plans and revisions to treatment plans. The control unit 320 may also provide information, power, and/or instructions to the hub 308 of the wearable article, or directly or indirectly via the hub, to signal source components of the management system.

The control unit 320 may further serve to output information to a computing device of one or more healthcare providers and/or the patient communication device. The control unit 320 may be in communication with one or more components of the wearable article 302, for example, via wired cables or wireless communication such as Bluetooth or ZigBee connections, and other communication mechanisms. In some implementations, the control unit 320 may communicate and interact with the hub 308 on the wearable article 302, which may transfer signals from the signal source components to the control unit 320.

When the patient is awake and mobile, the control unit remains in communication with the components, such as the hub 308. During awake periods, the control unit may be transported by the patient by various modes, such as in a pack or purse carried by the patient, on a belt, by a strap over the shoulder, or additionally by further adapting the wearable article 302, and so on. During a sleeping period of the patient, the control unit 104 may be placed at a proximal distance away from the patient, such as on a bed or table. In some implementations, the control unit may be removeably integrated with the wearable article.

Based on the findings from data analysis of the data management device, further treatment may be determined to be warranted. In some implementations, the processor 230 may determine treatment based on additional information as well, such as patient medical history data, event history data, etc. The processor 230 may activate discharge circuit 270 to deliver an appropriate shock treatment to the patient. In some implementations, when the determination is to shock, an electrical charge pulse is delivered to the patient. Delivering the electrical charge is also known as discharging. Shocking can be for defibrillation, pacing, and so on.

The hub 308 receives data from the external sensor 342 and transfers instructions or activation signals to the external sensor 342 via wired cable connection 334 and/or a wireless communication mechanism. In some implementations, an external sensor 342 may transfer signals directly to control unit 320 rather than to hub 308. In some implementations, the control unit 320 may receive signals or data from the hub 308 process the data and transmit the processed patient data to the data management device (not shown).

Various communication mechanisms may be employed between components of the management system, such as among wearable article components including hub 308, electrodes 304, and additional sensor components 350 and between external components such as control unit 320 and external sensor 342. Such components may be attached via wires to certain other components. Other communication mechanisms between components are possible. For example, some implementations may employ a conductive material in the wearable article 302, such as conductively enhanced fabric comprising the wearable article, to provide for passage of electrical communication signals from component to component through the wearable article.

Wireless communication mechanisms may include Bluetooth, radiofrequency, Zigbee, Wi-Fi, near field communication (NFC), infrared communication, GPS, and other wireless communication technologies. Wireless communication may employ security protocols to protect health signal, data, and information.

Electrodes 304 are removably fixed to an inside of the wearable article 302 to make contact with the skin of the patient directly or through a conductive medium, such as an electrolyte. The electrodes 304 may be electrically coupled to control unit 320 or to the hub 308 via electrode cable 322. The electrodes 304 may be functional as both therapy and monitoring electrodes, or just monitoring electrodes without therapy functionality. Electrodes 304 may be configured to produce electrical signals for ECG data and/or respiration data, e.g., AC signals for respiratory impedance determination, or DC signals.

Various sensors, such as electrodes 304, can be configured to be worn by the patient in a number of ways. The wearable article 302 can be configured to be worn by the patient so as to maintain the electrodes 304 on the body of the patient, while the patient is moving around, etc. The electrodes 304 can be maintained on the body attachment to the skin of the patient, simply pressed against the skin directly or through garments, etc. In some implementations the electrodes 304 are not necessarily pressed against the skin, but become biased that way upon sensing a condition that could merit intervention.

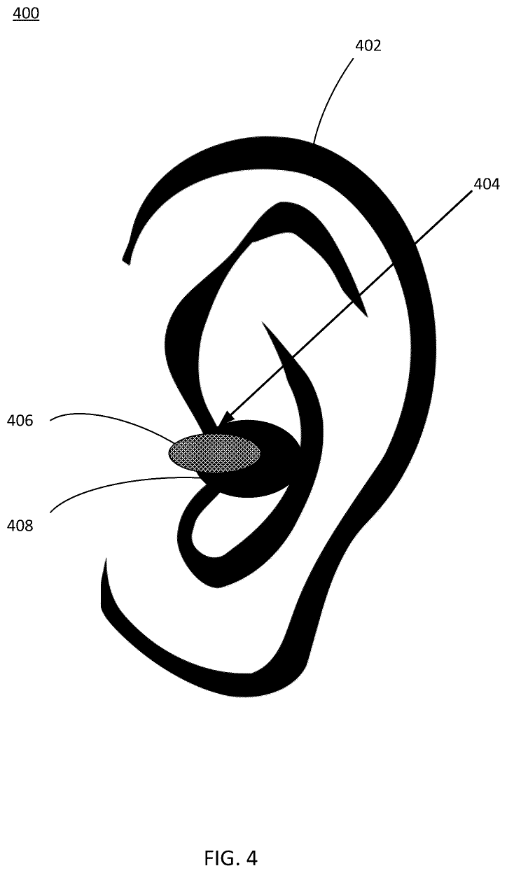

FIG. 4 shows an example stimulation device 400 installed in an ear 402 of a patient. The stimulation device 400 is coupled to a tragus portion 404 of the ear 402 by a clip 408 to contact electrode 406 to the surface of the tragus portion 404. Other components of the stimulation device 400 are possible, such as wires, receivers, etc. The stimulation device 400 is shown for illustration purposes of one type of device and other types of devices are possible that couple to other parts of the patient.

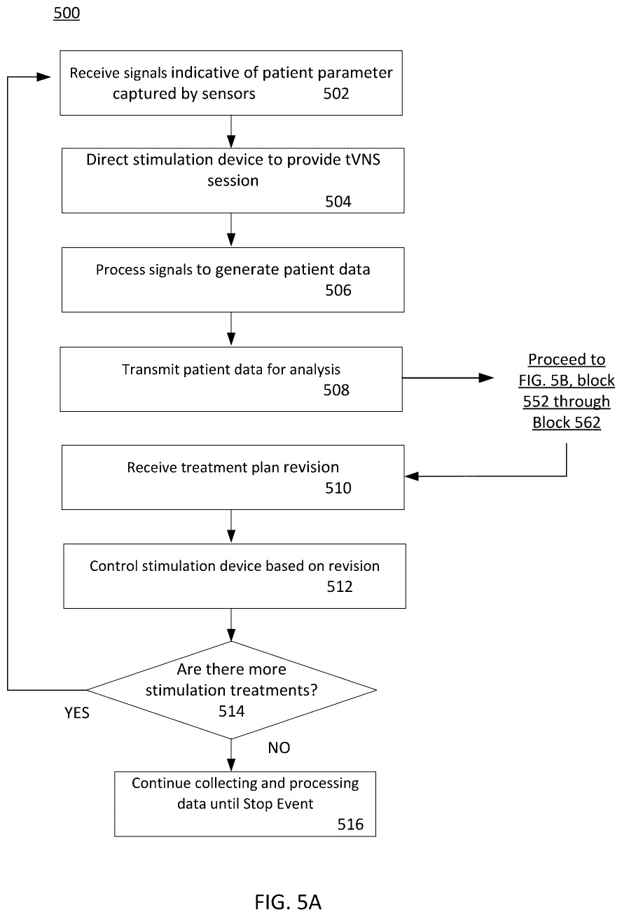

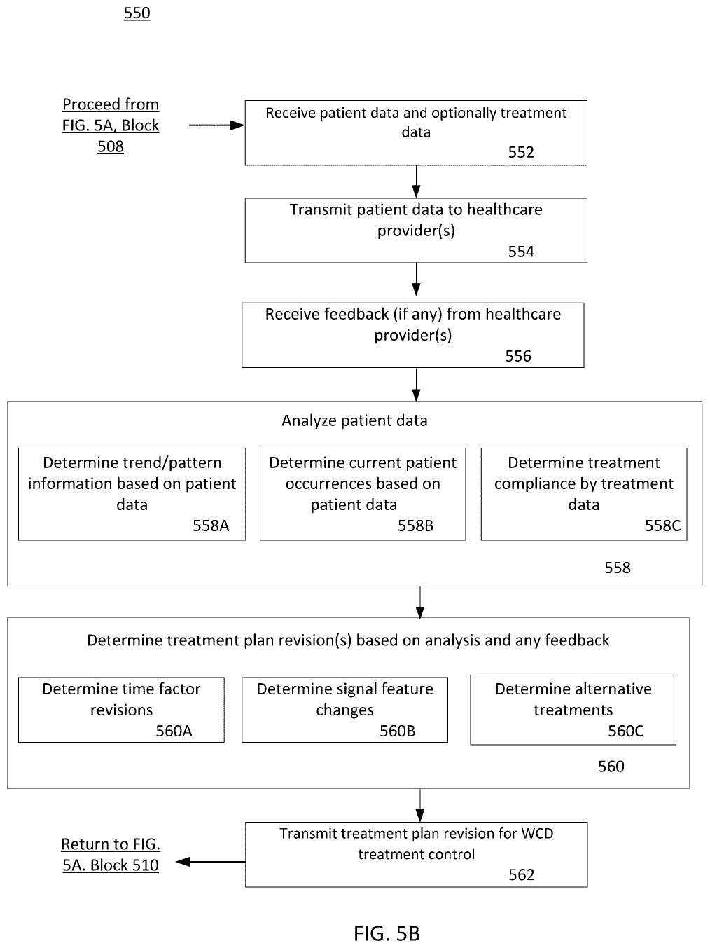

FIGS. 5A and 5B are combined to show flowcharts of an example VNS treatment management process for treatment of a health issue of a patient. In some implementations, the process is directed specifically to tVNS treatment of POTS of a patient using a stimulation device coupled to a tragus portion of an ear of the patient and using a torso fitted garment. In other implementations, the tVNS can be provided to treat other conditions or combinations of health issues, such as, for example, epilepsy, depression, heart failure and/or autoimmune and chronic inflammatory disorders including multiple sclerosis, headache, pain, Alzheimer's disease and inflammation as mentioned in Johnson R, Wilson C. A review of vagus nerve stimulation as a therapeutic intervention. J Inflamm Res 2018; 11: 203-213.

The treatment management process is performed by components of the medical management system, such as 100 of FIG. 1. FIG. 5A shows data collection and control steps of the VNS treatment management process 500.. For example the steps in FIG. 5A may be performed by WMD, such as 104 in FIG. 1 FIG. 5B shows data analysis and treatment plan generation process 550. For example, the steps in FIG. 5B may be performed by data management device 120 in FIG. 1.

With regard to FIG. 5A, in block 502, the WMD receives from sensors, signals that indicate various patient parameters. The signals are relevant to the health issue of the patient and/or the treatment. In some implementations, such as for treatment of POTS, the patient data may include a heartbeat rate and body position of the patient.

In block 504, the WMD directs a stimulation device to perform a VNS treatment session on the patient according to a treatment plan. The tVNS pulses can be administered to the patient via electrodes that attached to an ear of the patient to stimulate the auricular branch vagus nerve. In other embodiments, the tVNS therapy can be delivered to other parts of the patient's body (e.g., the patient's neck for the cervical branch vagus nerve).

In some implementations, the control by the WMD is for a duration length of time for the session, such as the WMD activating the stimulation device to commence a treatment session at a particular time of day, and then the WMD turning off the stimulation of the stimulation device at a session ending point. The session time may be for an hour to several hours or for a minute to several minutes, e.g., 1-10 minutes, and more particularly 4 minutes. The time factors may also include a timing pattern for a session such as continuous stimulation or periodic stimulation times. For example, the session may be controlled for stimulation to be performed continuously for one hour. In other cases, the stimulation may be a pulse pattern with stimulation for certain minutes, pause stimulation for some minutes, and then repeat the stimulation pattern for a particular session length of time. In some implementations, stimulation sessions by the stimulation device may be activated and/or deactivated by user input, such as a voice command, touch command, user gesture, etc., to start or end treatment. In this manner, a treatment session may be initiated or discontinued by the user as desired.

In some cases, the WMD control of the stimulation device may be directed to just a time factor, e.g., when to start/end the session or length of the session, and the stimulation device may use presets to determine signal features such as frequency, voltage, current and waveform of the signal. In other implementations, the WMD control may also specify one or more signal features.

In block 506, the signals from the sensors are processed into patient data. Processing may include extracting signals that are deemed by the WMD as relevant to the health issues, treatment plan and/or treatment sessions of the patient. Processing may also include formatting and packaging the data for transmission to the data management device.

In block 508, the patient data is transmitted to the data management device and the management process proceeds to FIG. 5B, block 552. In some implementations, treatment data is also processed by the WCD to reflect operations of the stimulation device or administration of other treatments. For example, recorded data of successful administration of electrical pulses at a particular time, duration, and signal features may be provided. In some cases, particular patient data may also reflect properly conducted alternative or supplemental treatments, such as a patient exercising, diet, fluid intake, taking medications, as specified in the treatment plan. A supplemental other treatment type may include an exercise regime and sensors may detect body motion data by an accelerometer to determine whether the planned exercise is performed by the patient. Treatment data reflects the carrying out of a treatment, and patient data includes effects of treatment and circumstances surrounding treatment.

In block 552, patient data which was processed by the WMD in block 506 is received by the data management device. In some cases, treatment data may also be received by the data management device that represents administration of treatment sessions. In some implementations, the patient data/treatment data from the WMD is transferred to a patient communication device, which in turn transmits the patient data to the data management device. In other implementations, the patient data is received directly from the WMD.

In block 554, in some implementations, the data management device may transfer at least a portion of the patient data to one or more healthcare provider devices. At times, it may be unnecessary to provide patient data to a particular healthcare provider. In some implementations, the data management device may provide treatment plan revisions from block 560 to the one or more healthcare provider devices.

In block 556, any feedback from the healthcare provider devices may be received. In some circumstances, an immediate decision from the data management device is required giving insufficient time or unnecessary to consider feedback from a healthcare provider. In such cases, the process may proceed to block 558 without considering any such feedback.

In block 558, patient data is analyzed according to analysis criteria. Examples of analysis criteria that may be employed are shown in subblocks 558A-558C. In subblock 558A patient data may be analyzed to determine trend/pattern information. In subblock 558B patient data may be analyzed to determine current patient occurrences and situations. In subblock 558C treatment data may be also analyzed to determine treatment compliance with a current treatment plan. The treatment data may include operations of a stimulation device and/or patient data that reflects treatment compliance. In some cases, analysis criteria may include an effectiveness threshold for which meeting the threshold amounts to effectiveness or ineffectiveness of the treatment. In still some cases, patient data may continue to be captured and analyzed during a post-treatment period of time to determine if an initially successful treatment has diminished past an effectiveness threshold to determine patient regression or to determine secondary health effects (e.g., undesirable side effects of treatment).

In some implementations a time course for the patient is plotted against significant patient data/treatment data/system that indicates a benefit and/or disadvantage of a time factor for aspects of the treatment plan. For example, the treatment plan may be revised to avoid stimulation sessions during certain times such as patient exercise above a threshold vigor, likely to be driving or sleeping. Other times for treatment sessions may be beneficial considering the patient's daily routine and sessions may be scheduled to occur during those times.

In some implementations, weights may be attached to particular types of patient data and analysis criteria to factor in data or criteria that are more significant to a particular treatment, patient, and/or health issue. Various combinations of analysis criteria, e.g., 558A-558C and other ways of analyzing the patient data and/or treatment data are possible.

In some implementations, analysis of patient data may reveal a trend or pattern as in block 558A, for example, that can be determined based on ongoing currently acquired patient data or a comparison of current patient data to stored historical data. Trends can be particularly useful for formulating the treatment plan or making revisions to the plan. Once a trend is detected, it can be stored and/or reported via a communication link, along with, perhaps, a warning to the patient via patient communication device 110 and/or and interface of the WMD 104.

In block 560, the analysis is used to determine treatment plan revisions, for example, as shown in subblocks 560A-560C. In some implementations, feedback from healthcare professionals may also be considered in generating treatment plan revisions. In subblock 560A time factor treatment revisions may be determined, such as a change in duration length of stimulation sessions. In subblock 560B signal feature changes for the stimulation device, such as electrical pulse strength may be determined. In subblock 560C a determination may be made that alternative including supplemental treatments may be warranted. For example, revisions may be made to promote patient compliance of the treatments. Various combinations of revisions, e.g., 560A-560C and other types of treatment plan revisions are possible.

In some cases, post-treatment period monitoring (e.g., as described in block 516 below) may indicate a patient regression or additional health issues that may require a second treatment plan. Such second treatment plan may be formulated by the data management system and rolled out (transmitted and treatments controlled) in a manner similar to carrying out the first treatment plan or revisions.

In block 562, the treatment plan revisions or a second treatment plan are transmitted back to the WMD. The management process returns to FIG. 5A, blocks 510-516. In block 510, the WMD receives a revision of the treatment plan devised by the data management system in FIG. 5B.

In block 512, the WMD controls the stimulation device and may control other treatment devices, according to the received revision. For example, a time of day for treatment sessions may be changed.

In decision block 514 it is determined whether there are additional treatment sessions required of the stimulation device according to the treatment plan revision. If there are more stimulation sessions, the process returns to 502 to continue receiving sensor signals for continued control of the stimulation device.

If there are no more stimulation sessions, the process moves to block 516 to continue monitoring the patient for a post-treatment period until a stopping event occurs. For example, the post-treatment monitoring period may be predefined to last for a period of time, such as 1-6 months after the last treatment session is performed. In some cases, the stopping event may include a determination of patient regression that the beneficial effect of the prior treatment plan has diminished past an effective threshold. A next/second treatment plan (which may mimic the first treatment plan or revisions thereof) may be generated and communicated to the WMD to be initiated, such as repeating the process 500, change aspects of the treatment plan by the stimulation device, add or remove a supplemental or different treatment types, or discontinue with all treatments.

The methods of FIGS. 5A and 5B described herein can be performed via software, hardware, and combinations thereof. The process may be carried out in software, such as one or more steps of the process carried out by the medical management system. Although the description has been described with respect to particular implementations thereof, these particular implementations are merely illustrative, and not restrictive.

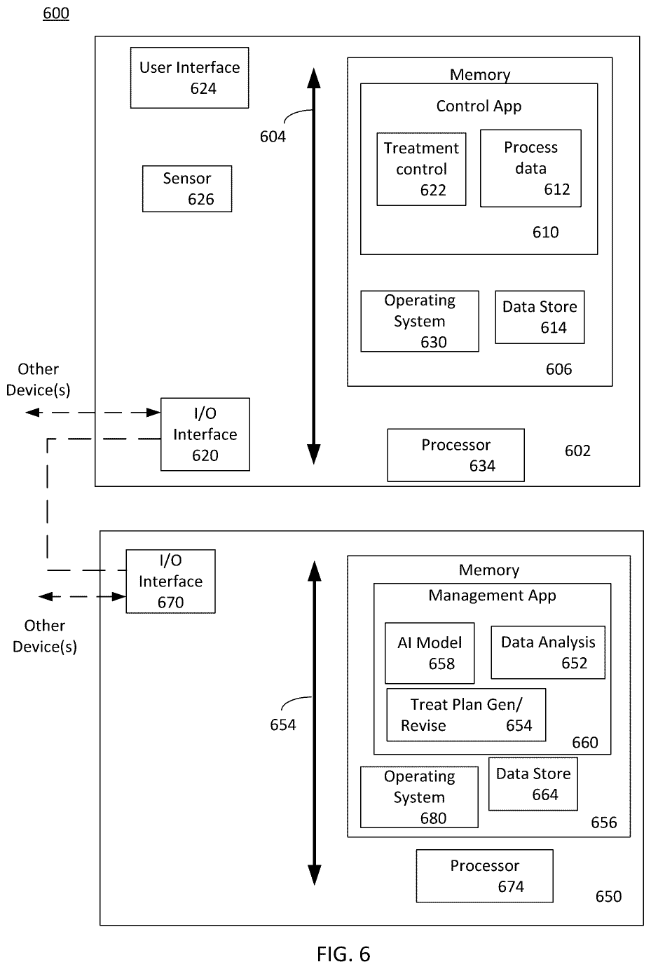

FIG. 6 is a block diagram of some medical management system 600 including a WMD 602, such as WMD 104 in FIG. 1 in communication via input/output interface 620 with an input/output interface 670 of a data management device 650, such as data management device 120 in FIG. 1. The input/output interface 620 and input/output interface 670 may be configured to also communicate with other devices, such as a patient communication device and healthcare provider devices.

The various elements of the WMD 602 and data management device 650 are shown in FIG. 6 as discrete/separate elements for purposes of illustration and explanation. According to some implementations, it is possible to combine some of these elements into a single element or device, while in other implementations of the medical management system, these elements may be distributed across a network such as in a cloud computing network. For example, functions of the medical management system may be performed by one or multiple WMD's and/or data management devices in a distributed network to provide information to other various devices of the management system and/or third party devices and perform other tasks as a service. Components of WMD 602 may be connected via signal lines to bus 604 such as processor 634 coupled to memory 606. Components of data management device 650 may be connected via signal lines to bus 654 such as processor 634 coupled to memory 65.6

The processor 634 of the WMD 602 may be implemented in a number of ways. Such ways include, by way of example and not limitation, digital and/or analog processors such as microprocessors and Digital Signal Processors (DSPs); controllers such as microcontrollers; software running in a machine; programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

The processor 634 processes instruction for execution within the WMD 602 including instructions stored in memory 606 or on the data store 614. The processor 930 may coordinate computing device components, e.g. applications, wireless or wired communication through interfaces, etc. In some implementations, multiple processors and buses 604 may be used.

Processor 634 may include, or have access to, a non-transitory storage medium, such as a memory 606. Such a memory 606 can have a non-volatile component for storage of machine-readable and machine-executable instructions. A set of such instructions can also be called a program or logic. The instructions, which may also referred to as “software,” generally provide functionality by performing methods as may be disclosed herein or understood by one skilled in the art in view of the disclosed implementations. In some implementations, and as a matter of convention used herein, instances of the software may be referred to as a “module” and by other similar terms. Generally, a module includes a set of the instructions so as to offer or fulfill a particular functionality. Implementations of modules and the functionality delivered are not limited by the implementations described in this document.

Memory 606 is shown for illustration purposes as a single memory, but may also be a collection of various types of primary, secondary and cache memories implemented in a number of ways by WMD 602. Such ways include, by way of example and not of limitation, volatile memories, Nonvolatile Memories (NVM), Read-Only Memories (ROM), Random Access Memories (RAM), magnetic disk storage media, optical storage media, smart cards, flash memory devices, any combination of these, and so on. Memory 606 is thus a non-transitory storage medium. Memory 606 can include programs for processor 634, which processor 634 may be able to read and execute. More particularly, the programs can include sets of instructions in the form of code, which processor 634 may be able to execute upon reading. Executing is performed by physical manipulations of physical quantities, and may result in functions, operations, processes, actions and/or methods to be performed, and/or the processor to cause other devices or components or blocks to perform such functions, operations, processes, actions and/or methods. The programs can be operational for the inherent needs of processor 634, and can also include protocols and ways that decisions can be made by data management device 600.

Memory 606 may be employed to store data in data store 614, such as patient data and system data. In some implementations, at least a portion of information may be stored on a disk drive or other computer readable storage device (not shown) within the WMD 602. Such storage device include a floppy disk device, a hard disk device, an optical disk device, or a tape device, digital cards, a flash memory or other similar solid state memory device, or an array of devices.

Processor 634 can be considered to implement a number of modules stored in memory 606, such as module components of control application 610. One such module can be a data processing module 612 to convert sensor signals into patient data. For example, the data processing module 612 may implement logic to perform the data processing steps described above, e.g., with regards to FIG. 5A. The module 612 may combine various patient data that may implicate a change in time factor for one or more treatment sessions, and/or a need for a different type of treatment, as analyzed by data management device 650.

The processor 634 can also implement modules to perform other functions for multiple purposes. For example, the treatment control module 622 may operate stimulation device and other treatment devices, or other devices employed for the data management system. Additional modules are possible to implement the described processes.

Generally, a module includes a set of the instructions so as to offer or fulfill a particular functionality. Implementations of modules and the functionality delivered are not limited by the implementations described in this document. The computer program may be tangibly embodied in an information carrier such as computer or machine readable medium, for example, the memory 606, storage device or memory on processor 634. A machine readable medium is any computer program product, apparatus or device used to provide machine instructions or data to a programmable processor.

The WMD 602 further includes the operating system 630. Any operating system 630, e.g., server OS, that is supports the data processing, analysis, control processes described herein performed by the WMD 602 may be employed.

User interface 624 can connect to interface devices for a user to communicate with the WMD 602 and management system 600, such as input devices (keyboard, pointing device, touchscreen, microphone, camera, scanner, sensors, etc.) and/or output devices (display devices, speaker devices, printers, motors, etc.). Some implementations can provide a microphone for capturing sound (e.g., as a part of captured images, voice commands, etc.), audio speaker devices for outputting sound, or other input and output devices.

Data management device 650 includes processor 674 that can be considered to implement a number of modules stored in memory 656 which may be implemented as modules of a medical management application 660. Data analysis module 652 performs analysis on patient data, treatment data, recommendation information from providers, etc., for example as described above. Treatment plan module 654 may determine and generate a treatment plan based on analysis results of the data analysis module 652, including any revisions to existing treatment plans based, at least in part, on the patient data. In some implementations, one or more AI models 658 may be stored in memory 656 to be implemented in predicting treatment plans/revisions, as described above. Additional modules are possible to implement the described processes.

The data management device 650 further includes a memory 656, which can work together with processor 674. Data store 664 may store analysis criteria to be accessed and used by data analysis module 652. The above described functions of basic components of WMD 602 may be applied similarly to comparable components of data management device 650 including processor 634 corresponding with processor 674, memory 606 corresponding with memory 656, operating system 630 corresponding with operating system 680, and data store 614 corresponding with data store 664.

The devices and/or systems described in this document perform functions, processes and/or methods. These functions, processes and/or methods may be implemented by one or more devices that include logic circuitry. Such a device can be alternately called a computer, and so on. It may be a standalone device or computer, such as a general purpose computer, or part of a device that has one or more additional functions. The logic circuitry may include a processor and non-transitory computer-readable storage media, such as memories, of the type described elsewhere in this document. Often, for the sake of convenience only, it is preferred to implement and describe a program as various interconnected distinct software modules or features. These, along with data are individually and also collectively known as software. In some instances, software is combined with hardware, in a mix called firmware.

Moreover, methods and algorithms are described above. These methods and algorithms are not necessarily inherently associated with any particular logic device or other apparatus. Rather, they are advantageously implemented by programs for use by a computing machine, such as a general-purpose computer, a special purpose computer, a microprocessor, a processor such as described elsewhere in this document, and so on.

This detailed description includes flowcharts, display images, algorithms, and symbolic representations of program operations that may be provided within at least one non-transitory, tangible, computer readable medium for execution by the one or more processors. An economy is achieved in that flowcharts as in FIGS. 5A-5B are used to describe both programs, and also methods. So, while flowcharts described methods in terms of boxes, they also concurrently describe programs.

We claim:

1. A medical management system for managing vagus nerve stimulation (VNS) treatment for a health issue of a patient, comprising:
a wearable article configured for long term wear and health monitoring of the patient, the wearable article including:
a control unit configured to direct a stimulation device to provide first VNS sessions according to a first treatment plan;
one or more sensors configured to capture signals indicative of at least one patient parameter;
a data handling unit in communication with the one or more sensors to generate patient data from the signals related to the first treatment plan; and
an interface configured for transmitting the patient data for receipt by a data management device to conduct data analysis with respect to the first treatment plan,
wherein the interface is further configured for receiving a revision of the first treatment plan by the data management device, wherein the revision includes a time factor change for treatment and/or type of treatment change based, at least in part, on the data analysis, and
wherein the control unit is further configured to direct the stimulation device according to the revision.

2. The medical management system of claim 1, wherein the patient data includes treatment data indicating delivery characteristics of the first VNS sessions,
wherein the data analysis indicates a lack of patient compliance with the first treatment plan, and wherein the revision includes a change to the first treatment plan to address the lack of patient compliance.

3. The medical management system of claim 1, wherein at least a portion of the signals are captured during a post-treatment period after conclusion of the first treatment plan, and wherein the data analysis indicates a patient regression, and
wherein the interface is further configured to receive a second treatment plan from the data management device, including instructions for: repeating the first VNS sessions, providing second VNS sessions using a different stimulation device, and/or providing a supplemental other treatment type.

4. The medical management system of claim 3, wherein the control unit is further configured to direct the repeating of the first VNS sessions by the stimulation device or the providing of second VNS sessions by the different stimulation device, according to the second treatment plan.

5. The medical management system of claim 1, wherein the change in the time factor is determined based, at least in part, on the signals that indicate a patient state, patient action, and/or environment.

6. The medical management system of claim 1, wherein the time factor includes at least one of a VNS session duration, a time of day for at least one first VNS session, a period of time for a course of multiple VNS sessions, and discontinuing of the first VNS sessions.

7. The medical management system of claim 1, further comprising a communication component to transmit a notification of an impending session of the VNS first sessions prior to controlling the providing of the impending session.

8. A method by a wearable medical device (WMD) configured for long term wear on a patient to manage vagus nerve stimulation (VNS) treatment for a health issue of a patient, the method comprising:
receiving signals indicative of at least one patient parameter, captured by one or more sensors of the WMD;
directing a stimulation device to provide first VNS sessions according to a first treatment plan;
processing the signals to generate patient data;
transmitting the patient data for receipt by a remote management device to conduct data analysis with respect to the first treatment plan;
receiving a revision of the first treatment plan generated by the remote management device, the revision including a time factor change for treatment and/or type of treatment change based, at least in part, on the data analysis; and
controlling the stimulation device according to the revision.

9. The method of claim 8, wherein the patient data includes treatment data indicating delivery characteristics of the first VNS sessions, and
wherein the data analysis indicates a patient compliance with the first treatment plan.

10. The method of claim 8, wherein at least a portion of the signals are captured during a post-treatment period after conclusion of the first treatment plan, and wherein the data analysis indicates a patient regression, and
the method further comprising:
receiving a second treatment plan from the remote management device, including instructions for: repeating the first VNS sessions, providing second VNS sessions using a different stimulation device, and/or providing a supplemental other treatment type.

11. The method of claim 10, further comprising:
controlling, by the WMD, the repeating of the first VNS sessions by the stimulation device or the providing of second VNS sessions by the different stimulation device, according to the second treatment plan.

12. The method of claim 8, wherein the change in the time factor is determined based, at least in part, on the signals that indicate a patient state including at least one of sleep, exercise, water exposure, consciousness, body position, body movement, seizure, and falling.

13. The method of claim 8, wherein the time factor includes at least one of a VNS session duration, a time of day for at least one first VNS session, a period of time for a course of multiple VNS sessions, and discontinuing of the first VNS sessions.

14. The method of claim 8, further comprising outputting a notice for the patient of an impending session of the VNS first sessions prior to controlling the providing of the impending session.

15. A medical management system for managing transcutaneous vagus nerve stimulation (tVNS) for treatment of Postural Tachycardia Syndrome (POTS) of a patient, the system comprising:
a stimulation device configured to emit electrical pulses to a portion of an ear of the patient;
a wearable medical device (WMD) comprising:
a wearable article configured for long term wear on a torso of the patient including one or more sensors configured to capture signals indicative of at least one patient parameter including a patient state;
a control unit configured to direct the stimulation device to provide first tVNS sessions according to a first treatment plan;
a data handling unit in communication with the one or more sensors to generate patient data from the signals related to the first treatment plan; and
an interface configured for transfer the patient data to a data management device for data analysis and to receive from the data management device, a revision of the first treatment plan including a time factor change based, at least in part, on the data analysis,
wherein the control unit is further configured to direct the stimulation device according to the revision.

16. The medical management system of claim 15, wherein the patient data includes a heartbeat rate and body position and the time factor includes a change in a session duration for treatment.

17. The medical management system of claim 15, wherein the revision includes a supplemental other treatment type including an exercise regime and the patient data includes body motion data by an accelerometer.

18. The medical management system of claim 15, wherein the time factor further includes at least one of a time of day for at least one first tVNS session, a duration for at least one tVNS session, a period of time for a course of multiple tVNS sessions, and discontinuing of the first tVNS sessions.

19. The medical management system of claim 15, wherein the WMD further comprising cardiac pacing components and the treatment plan includes outputting pacing pulses to the patient by the cardiac pacing components when the patient is determined to be standing from a sitting or lying position.

20. The medical management system of claim 15, wherein at least a portion of the signals are captured during a post-treatment period after conclusion of the first treatment plan,
wherein the data analysis indicates a patient regression, and
wherein the revision of the first treatment plan includes instructions for: repeating the first tVNS sessions, providing second tVNS sessions using a different stimulation device, and/or providing a supplemental other treatment type.
